(19) United States
(12) Patent Application Publication
(10) Pub. No.: US 2010/0179629 A1
(43) Pub. Date: Jul. 15, 2010

(54) MEDICAL IMPLANTABLE LEAD AND METHOD FOR MANUFACTURING THE SAME

(76) Inventors: Rolf Hill, Jarfalla (SE); Olof Stegfeldt, Alta (SE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/663,724

(22) PCT Filed: Dec. 21, 2007
(86) PCT No.: PCT/SE2007/001153

§ 371 Date: Dec. 9, 2009

(30) Foreign Application Priority Data
Jun. 28, 2007 (SE) PCT/SE2007/000634

(51) Int. Cl. A61N 1/05; H01R 43/00

(52) U.S. Cl. 607/126; 29/877

(57) ABSTRACT

In a medical implantable lead for monitoring and/or controlling of an organ inside a human or animal body, and a method for the manufacture thereof, two electrical conductors are each connected to a respective electrode for receiving or transmitting of electrical signals from or to the organ, and a tubular header is provided in a distal end of the lead. One electrode is positioned in the outermost distal end of the lead whereas the other is a ring electrode that is mounted by a coupling on the outside of the header. The coupling and the header are integrated into one unitary piece of an electrically insulating material having a coupling portion, and the coupling portion is adapted configured for quick fixing connection of the ring electrode to the header.

The invention relates to a medical implantable lead for monitoring and/or controlling of an organ inside a human or animal body, comprising two electrical lead conductors, each connected to a respective electrode for receiving or transmitting of electrical signals from or to the organ, and a tubular header in a distal end of the lead, wherein one electrode is positioned in the outermost distal end of the lead whereas the other is a ring formed electrode, which is mounted on the outside of the header by means of a coupling.

The invention also relates to a method for manufacturing of such a medical implanable lead.

### DESCRIPTION OF THE PRIOR ART

Medical implantable leads of the above kind are well known in the art. One common field of application is for monitoring and/or controlling of the function of a human or animal heart, in which case it is inserted into the body and attached with a distal end to the heart by means of a fixating member, such as a helix which can be screwed into the tissue. A proximal end of the lead is connected to an electronic device, for performing the monitoring and/or controlling, such as a pacemaker or an implantable cardiac defibrillator.

The most distal of the electrodes can be formed as a passive electrode, in which case it is formed in the end surface of the lead and bears against the surface of the heart while being fixed to the heart wall by means of fins, tines or the like, which during implantation engages the trabecular network inside the heart and subsequently are overgrown by a layer of tissue. The distal electrode can also be of an active type, which is used both for monitoring/controlling and for fixation in that the electrode is inserted into and is engaged with the tissue, such as for example a tip provided with barbs or a helix which is screwed into the tissue. The most proximal of the electrodes is formed as a ring on the outside of the lead and positioned on a suitable distance from the distal end. The distance from the distal end is determined by the desired amplitude of the signals and an acceptable interference level.

Normally, the ring electrode is positioned at the proximal end of a so called header, which is formed as a tubular and rigid housing positioned in the distal end of the lead and adapted to carry the electrodes and the fixating member, e.g. accommodating the helix inside an inner bore of the header. The most common way to accomplish the mounting of the ring electrode to the header, is to arrange a coupling member between the header and the ring electrode which is partly inserted into the tubular bore from the proximal end of the header and partly into the ring electrode. In this way a distance is achieved between the ring electrode and the distal electrode, which is suitable for most applications and the outer surface of the ring electrode can be mounted flush with the outer surface of the header or a sleeve covering the header such that the maximum diameter of the lead can be made advantageously small. The fixation of the ring electrode in relation to the coupling member is normally achieved by an adhesive, which has to result that the assembled header, coupling member and ring electrode have to be held immovable and fixed during the time for curing of the adhesive. This is a disadvantage since it has to effect that the manufacturing process of the lead has to be suspended for a while. Moreover, this manufacturing method of the lead is also disadvantageous in that there is not provided any fixed position for the ring electrode in relation to the header. This has to effect that the position and orientation of the ring electrode in relation to the header can vary and the production yield loss of this kind of leads is comparatively high due to failures when interconnecting the ring electrode and the header.

Normally, the header as well as the coupling member are manufactured of an electrically conducting material, such as titanium, which means that a separate electrically insulating layer must be provided since the ring electrode must be electrically insulated in relation to the header. However, in prior art is also known headers made of an electrical insulating material, such as PEEK (polyetheretherketone). In this case there is no need for an electrical insulation between the ring electrode and the header. In the prior art it is also known to place the ring electrode on the outside around and bearing directly against a header of an electrically insulating material. In this way it is possible to arrange a defined position for the ring electrode on the header. However, an adhesive must be used to finally fixate the ring electrode on the header, which is a disadvantage in that the adhesive has to be applied carefully in a correct position, in order to ensure proper function of the completed lead, and requires some time for curing during which time the ring electrode may be dislodged from its position. Another disadvantage with such a design of the lead is that the ring electrode must be connected electrically, by means of a separate conductor, to a conductor in the lead. This adds additional components and manufacturing steps to the manufacturing process.

### SUMMARY OF THE INVENTION

An object of the present invention is to improve medical implantable leads according to prior art and provide a medical implantable lead which can be manufactured to a low cost.

The invention also relates to a method for manufacturing of a medical implantable lead, having essentially the same object as above.

The above object is achieved in accordance with the present invention by a medical lead for monitoring and/or controlling an organ inside a human or animal body, and a method for the manufacture thereof, wherein two electrical conductors are each connected to a respective electrode for receiving or transmitting electrical signals from or to an organ, and a tubular header is provided in a distal end of the lead. One of the electrodes is positioned in the outermost distal end of the lead, whereas the other of the electrodes is a ring electrode that is mounted by a coupling on the outside of the header. The coupling and the header are integrated into one unitary piece of electrically insulating material having a coupling portion, and the coupling portion is configured for quick fixing connection of the ring electrode to the header.

The basis of the invention is the insight that the above object can be achieved by manufacturing the header of an electrically insulating material and to integrate the coupling member and the header into one unitary piece, wherein the coupling member is formed as a coupling portion in the header and formed in such a way that the ring electrode can be instantly fixed to the header by means of a quick fixating connection. Even though it is possible to also use an adhesive for increased bonding strength and fluid tight sealing of the lead, it is in this way possible to fix the ring electrode instantly without having to wait for the adhesive to cure before proceeding with subsequent manufacturing steps.

Within this overall inventive idea, the invention may be realized in many different ways within the scope of the associated claims. For example the quick fixating connection may be formed in various ways. In two hereinafter described embodiments of the invention, the quick fixating connection is formed as a clamp connection, wherein the ring electrode is clamped around a coupling portion of the header. In the described embodiments, the clamp connection is carried out by punching locking tabs from the ring electrode, which engage in a coupling portion in form of recesses in the outer surface of the header and lock the ring electrode in relation to the header. This kind of clamp connection is advantageous in so far as it prevents the ring electrode from being displaced in the longitudinal direction as well being rotated in relation to the header. However, the clamp connection can be carried out also in other ways, such as a clamp connection where the circumference of the ring electrode is crimped around and pressed towards the header. A quick fixating connection in form of a clamp connection is also advantageous in so far as it easily can be used to locate the ring electrode with different desirable distance from the distal tip of the medical implantable lead, as is illustrated in two different embodiments hereinafter.

In the described and illustrated clamp connection embodiments, the ring electrode is formed such that a proximal sleeve portion, in the mounted state, reaches over an electrical conductor, which suitably is in form of an outer wire coil and which provides electrical connection to the proximal end of the medical implantable lead, such that the ring electrode also can be electrically connected to the outer wire coil by means of a clamp connection. In this way no separate connector is required for connection of the ring electrode to the outer wire coil. Preferably, the clamp connection of the ring electrode to the header as well as to the outer wire coil, is performed in the same operation. Also preferably, the connection of the outer wire coil to the header is performed in the same operation and with the same clamp connection as when connecting the ring electrode to the outer wire coil. However, it is to be understood that it would also be possible to perform the clamp connection of the ring electrode to the header and to the outer wire coil in separate operations and the outer wire coil can be attached to the header by some other means, such as by welding, for increased strength.

In another hereinafter described and illustrated embodiment, the quick fixating connection is formed as a snap fit attachment, such that the coupling portion in the proximal end of the header is provided with a circumferential groove on the outside, into which an inner, circumferential bead in a distal end of the ring electrode can go into engagement by deformation of the material in the ring electrode.

However, the snap fit attachment could be formed in many other ways and it would also be possible to arrange a quick fixating connection in form of a thermo connection between the ring electrode and the header.

In the described and illustrated embodiments of the invention, the fixating member for fixating the lead to an organ inside a body, is formed as a helix, which is accommodated inside a bore in the header and which can be screwed out from the distal end and into the tissue of an organ. It is to be understood, however, that the fixating member can be formed in many other ways, such as for example as a sharp tip provided with barbs to be penetrated into the tissue or as fins or tines to be engaging the trabecular network inside a heart and to be subsequently overgrown by tissue. The lead can also be adapted for monitoring and/or controlling of other organs than a heart inside a body.

### BRIEF DESCRIPTION OF THE DRAWINGS

FIG. 1 is a longitudinal section through a distal end of a lead according to the prior art.

FIG. 2 is a longitudinal section through a distal end of a lead according to a first embodiment of the invention.

FIG. 3 is an enlarged detail, in longitudinal section, of the connection between the ring electrode and the header according to the first embodiment, in a disassembled state.

FIG. 4 is a partly exploded perspective view of the lead according to FIG. 3.

FIG. 5 is a longitudinal section through a distal end of a lead according to a second embodiment of the invention, in an unclamped state.

FIG. 6 is a longitudinal section through the embodiment of FIG. 6, during clamping.

FIG. 7 is a longitudinal section through a completed lead according to FIGS. 5 and 6.

FIG. 8 is a longitudinal section through a lead according to a third embodiment of the invention.

### DESCRIPTION OF THE PREFERRED EMBODIMENTS

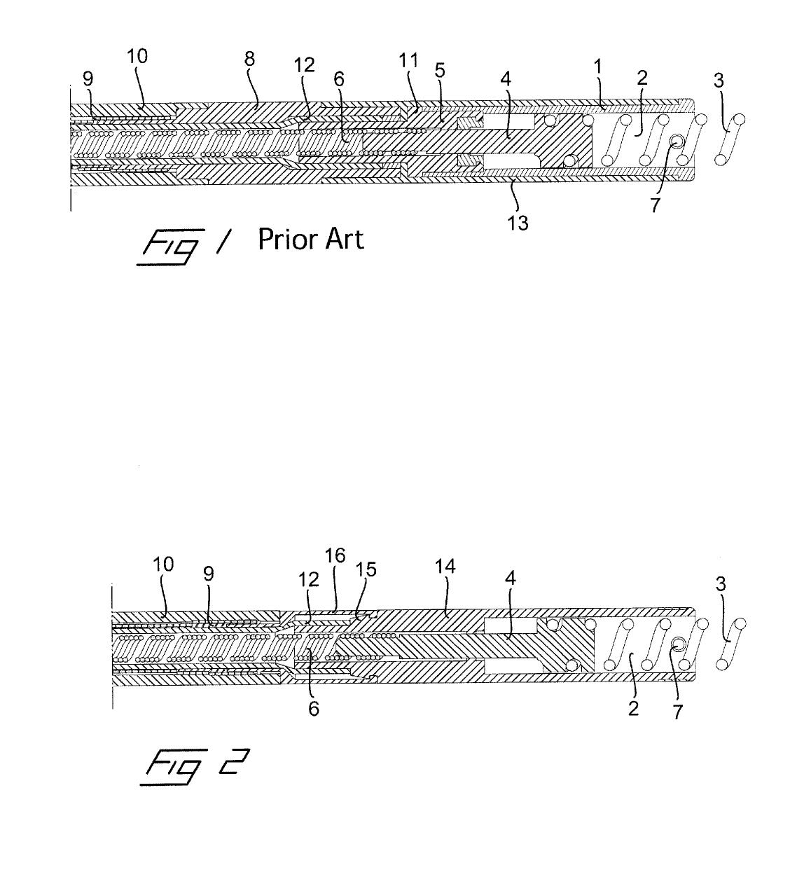

Reference is first made to FIG. 1 of the drawings, in which is illustrated a prior art embodiment of a distal end of a medical implantable lead. In the most distal end the lead comprises a tubular header 1 of an electrically conducting material such as titanium. In a distal bore 2 of the header, a helix 3 is accommodated, which is illustrated in a partially projecting state. The helix is mounted on a distal end of a shaft 4, which in its turn is journalled in a coupling member 5 such that it can be rotated as well as displaced in a longitudinal direction in relation to the coupling. The rotation of the helix 3 is actuated from the proximal end of the lead by rotation of an inner wire coil 6, which also functions as an electrical conductor from the proximal to the distal end. The helix 3 is in engagement with a post 7 on the inside of the header 1, such that when the inner wire coil 6 is rotated, the helix will be screwed out and advanced forward out from the header, which will have to effect that also the shaft 4 and the inner wire coil 6 will be advanced forward. When implanting the lead into a body, the helix is screwed out and into an organ such that the distal end is attached to the organ and the helix will function as an electrode in the organ. A second electrode is arranged in the proximal end of the header in form of a ring electrode 8, which is in electrical contact with the proximal end by means of an outer wire coil 9. Outside of the outer wire coil, an outer sleeve 10 is applied for protection of the lead.

Besides the function of rotationally bearing the shaft 4, the coupling member 5 also has the function of mounting and fixating the ring electrode 8. For this purpose, the coupling member 5 is formed with an intermediary section 11 having a comparatively large diameter, namely a diameter being equal to the outer diameter of the header 1, and end portions having a comparatively small diameter. When assembling the lead, one of the end portions of the coupling member is inserted into the header, as is illustrated in the drawing, and is interconnected by welding to the header, since also the coupling member is metallic. The other of the end portions is utilized for bearing of the ring electrode 8. However, since the header 1 and the coupling member 5 are metallic, they will have the same electrical potential as the shaft 4 and the helix 3. Accordingly, the ring electrode must be electrically insulated from the coupling member 5, which will have a different electrical potential. This is achieved by means of an inner sleeve 12, of silicone or the like, which surrounds the inner wire coil 6 and is extended up over a proximal end portion of the coupling member. The ring electrode 8 is positioned over the inner sleeve 12 and will accordingly be electrically insulated in relation to the coupling member 5, the header 1, the shaft 4 and the helix 3. However, the distal end of the ring electrode, is not allowed to come into electrical contact with the proximal end of the large diameter portion of the coupling member. Accordingly, when fixing the ring electrode to the coupling member by means of an adhesive, the ring electrode has to be held immovable, preferably in a fixture, in relation to the coupling member together with the header, for as long time as until the adhesive has been cured and the ring electrode is fixed. This means that the assembling of the lead has to be suspended for a while which is a disadvantage for the work flow when assembling the lead. Moreover, since there is no fixed position for the ring electrode on the coupling member, it frequently happens that the distance between the distal end of the ring electrode and the proximal end of the large diameter portion of the coupling becomes too small, in which case there is a risk for shortcircuiting, or too large in which case it is a risk that the assembled header, coupling and ring electrode does not fit with the rest of the lead components. It also happens that the header and the ring electrode becomes misaligned in relation to each other or that the insulating inner sleeve becomes damaged when passing the distal end of the ring electrode over the coupling member and the inner sleeve. All of these situations may lead to a rejection of the lead. Moreover, since the header is electrically conductive and has the same electrical potential as the distal electrode, i.e. the helix, it has to be covered by an electrically insulating layer in the form of a header sleeve 13, of e.g. silicone, which normally is bonded directly onto the header such that the adhesive also penetrates into the gap between the proximal flange of the ring electrode and the large diameter portion 11 of the coupling member.

As is evident from the above description, the distal end of a medical implantable lead according to prior art, is composed of many different components and several different assembling steps are required for the manufacturing. Accordingly, it is desirable to provide a medical implantable lead which contains less component parts and requires fewer assembling steps for manufacturing, in order to reduce costs.

Now reference is made to FIGS. 2-4 for description of a first embodiment of a medical implantable lead according to the invention. Here the coupling member and the header are integrated into one unitary piece and made of an electrically insulating material, e.g. of PEEK, in the form of a header 14 having a coupling portion 15 in the proximal end. Moreover, the proximal coupling portion is formed with a quick fixating connection, which allows for an instant connection between the coupling portion and a properly adapted ring electrode 16. More precisely, the coupling portion of the header is formed with a circumferential groove 17 in its outer surface, whereas the ring electrode is formed with an inner, circumferential bead 18 in its distal end, such that the ring electrode can be snap-fit connected to the header. To facilitate the snap-fit engagement, the header is formed with an inclined surface 19 proximal to the groove 17, such that the bead 18 of the ring electrode, upon pressing the ring electrode and the header axially towards each other, may slide over the inclined surface 19, while slightly deforming the ring electrode, and snap into the groove.

The header 14 according to the invention will serve several purposes. It will accommodate the helix inside the header bore 2 such that the helix may be completely contained inside the bore during inserting of the lead into the body and may be screwed out from the distal end and into the tissue for attaching the lead to an organ. Moreover, the header will provide for bearing of the shaft 4 and allow rotation as well as longitudinal displacement of the shaft. The header also provides, as already described, for means for quick fixing of the ring electrode to the header. Finally, the header provides for electrical insulation of the inner wire coil 6, shaft 4 and helix 3 from the ring electrode 16 and the outer wire coil 9. Since the ring electrode 16 is supported by the header 14 and the inner sleeve 12, which is extended over the outside of a proximal portion of the header, no additional insulating components are required between these parts.

The medical implantable lead according to the invention, will facilitate assembling of the lead and reduces the number of components required. For example, no separate coupling member is required and since the header is manufactured of an electrically insulating material, no additional insulating layer, as the layer 13 in FIG. 1, is required on the outside of the header. Moreover, the lead can be manufactured with an increased precision which reduces production yield losses. All these factors allows for manufacturing of medical implantable leads to a lower cost.

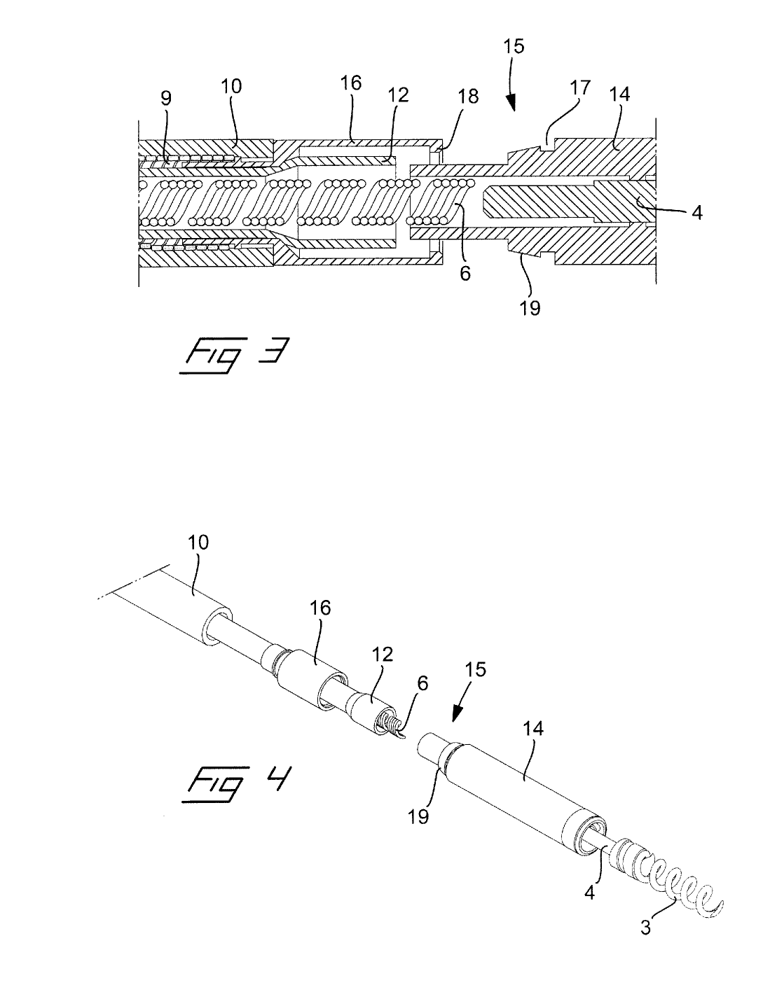

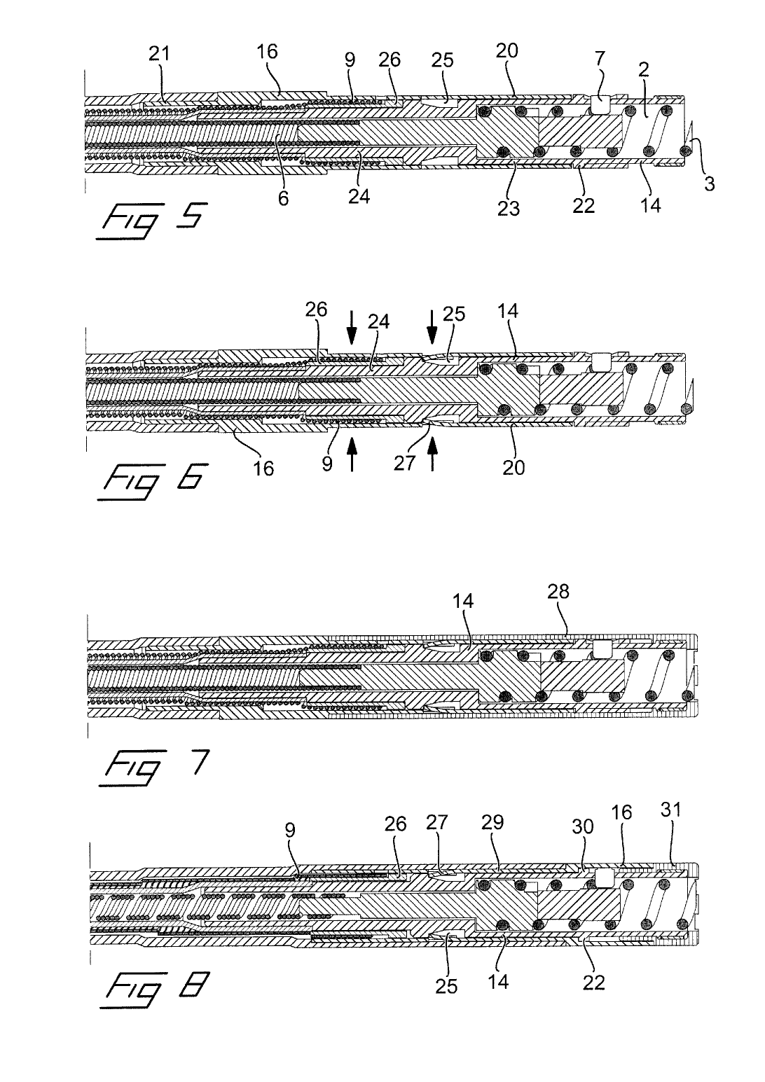

Reference is made to FIGS. 5 to 7, in which are illustrated a second embodiment of the invention. In this embodiment the attachment between the header sleeve 14, being made of an electrically insulating material, and the ring electrode 16, is performed by means of a clamping connection. More precisely, the ring electrode 16 is provided with a distal sleeve portion 20 and a proximal sleeve portion 21 having slightly smaller outer diameters than the actual ring electrode 16. The header sleeve 14 in its turn, has an enlarged diameter portion 22 near its distal end, an intermediary support portion 23 for the distal sleeve portion 20 of the ring electrode, and a proximal support portion 24 with reduced diameter for the outer wire coil 9. The intermediary support portion 23 is formed with a coupling portion in form of recesses 25 around its periphery. When mounting the ring electrode 16 onto the header sleeve 14, the ring electrode 16 is slid onto the header sleeve 14 from its proximal end, until the end of the distal sleeve portion 20 abuts a proximal end surface of the enlarged diameter portion 22 of the header sleeve. Subsequently, the outer wire coil 9 together with a crimping sleeve 26 is inserted between the inner periphery surface of the ring electrode 16 and the proximal support portion 24 of the header sleeve until the distal end of the crimping sleeve 26 abuts a proximal end of the intermediary support portion 23. Thereafter, the assembly is positioned in a clamping tool by means of which clamping forces are applied over the recesses 25 and the proximal support portion 24, as is illustrated by the arrows in FIG. 6. This has to effect that locking tabs 27 will be punched out from the distal sleeve portion 20 of the ring electrode 16, which will be bent down into the recesses 25 and lock the ring electrode in relation to the header sleeve, and a proximal portion of the distal sleeve portion 20 will be pressed and crimped around the proximal support portion 24 such that the outer wire coil 9 and the clamping sleeve 26 will be pressed towards and secured in relation to the proximal support portion 24 by means of the clamp connection of the ring electrode 16. Finally, an outer protective sleeve 28 of an electrically insulating material, is arranged over the distal end of the header sleeve 14 and the distal sleeve portion 20 of the ring electrode, as is illustrated in FIG. 7.

In FIG. 8 is illustrated a third embodiment which, similar to the embodiment illustrated in FIGS. 5 to 7, is formed with a quick fixating connection in form of a clamping connection. In this embodiment, however, the electrode surface of the ring electrode 16 is positioned close to the distal end of the header sleeve. This is accomplished by forming the ring electrode 16 with a proximal sleeve portion 29 which, at a transition section to the electrode surface, is formed with a stop surface 30 facing towards the distal end, which abuts against an enlarged diameter portion 22 of the header sleeve 14. The raised portion forming the electrode surface of the ring electrode 16, rests partly on the enlarged diameter portion 22 and partly on a silicone cap 31 in the end of the header sleeve. In all other respects, this embodiment is similar to the embodiment relating to the FIGS. 5 and 6, i.e. the header sleeve is provided with recesses 25 in the header for forming of locking tabs 27 in the ring electrode and the outer wire coil 9 is attached to the header sleeve by clamping the ring electrode around it and a clamping sleeve 26.

Although modifications and changes may be suggested by those skilled in the art, it is the intention of the inventors to embody within the patent warranted heron all changes and modifications as reasonably and properly come within the scope of their contribution to the art.

1.-17. (canceled)

18. An implantable medical lead for monitoring and/or controlling an organ inside the body of a living subject, comprising:
a lead body having a proximal end and an opposite distal end;
two electrical conductors connected to respective electrodes for receiving or transmitting electrical signals from or to the organ;
a tubular header in a distal end of said lead body;
one of said electrodes being positioned at an outermost distal location in said distal end of said lead body, and the other of said electrodes being a ring electrode mounted outside of said header by a coupling; and
said coupling and said header being integrated into a single unitary piece of electrically insulating material with said header formed with a coupling portion, said coupling portion being configured for quick fixing connection of the ring electrode to the header.

19. An implantable medical lead as claimed in claim 18 wherein said quick fixing connection is a clamp connection.

20. An implantable medical lead as claimed in claim 19 wherein said clamp connection comprises locking tabs punched from said ring electrode that engage recesses in said coupling portion in an outer surface of said header.

21. An implantable medical lead as claimed in claim 18 wherein said ring electrode comprises a sleeve portion extending over one of said electrical conductors and connected to said one of said electrical conductors by a clamp connection.

22. An implantable medical lead as claimed in claim 21 wherein said clamp connection also connects said one of said conductors to said header.

23. An implantable medical lead as claimed in claim 18 wherein said quick fixing connection is a snap fit connection.

24. An implantable medical lead as claimed in claim 23 wherein said snap fit connection comprises an outer circumferential groove in a proximal end of said header and an inner circumferential bead in a distal end of said ring electrode.

25. An implantable medical lead as claimed in claim 18 wherein said quick fixing connection is a thermo connection.

26. An implantable medical lead as claimed in claim 18 comprising, in addition to said quick fixing connection, an adhesive that forms a seal against fluids entering into an interior of said lead body.

27. A method for manufacturing an implantable medical lead for monitoring and/or controlling an organ inside the body of a living subject, comprising the steps of:
providing a lead body having a proximal end and an opposite distal end;
providing two electrical conductors connected to respective electrodes for receiving or transmitting electrical signals from or to the organ;
providing a tubular header in a distal end of said lead body;
positioning one of said electrodes at an outermost distal location in said distal end of said lead body, and forming the other of said electrodes as a ring electrode mounted outside of said header by a coupling; and
integrating said coupling and said header into a single unitary piece of electrically insulating material with said header formed with a coupling portion, and configuring said coupling portion for quick fixing connection of the ring electrode to the header.

28. A method as claimed in claim 27 comprising configuring said quick fixing connection as a clamp connection.

29. A method as claimed in claim 28 comprising forming said clamp connection with locking tabs punched from said ring electrode that engage recesses in said coupling portion in an outer surface of said header.

30. A method as claimed in claim 27 comprising forming said ring electrode with a sleeve portion extending over one of said electrical conductors and connected to said one of said electrical conductors by a clamp connection.

31. A method as claimed in claim 30 comprising, with said clamp connection, also connecting said one of said conductors to said header.

32. A method as claimed in claim 27 comprising configuring said quick fixing connection as a snap fit connection.

33. A method as claimed in claim 32 comprising forming said snap fit connection with an outer circumferential groove in a proximal end of said header and an inner circumferential bead in a distal end of said ring electrode.

34. A method as claimed in claim 27 comprising configuring said quick fixing connection as a thermo connection.

35. A method as claimed in claim 27 comprising, in addition to said quick fixing connection, providing an adhesive that forms a seal against fluids entering into an interior of said lead body.
